(19) United States
(12) Patent Application Publication
(10) Pub. No.: US 2014/0213559 A1
(43) Pub. Date: Jul. 31, 2014

(54) COMPOSITIONS AND TREATMENTS FOR DYSTROPHIES

(71) Applicants: Madeleine Durbeej-Hjalt, Lomma (SE); Virginie Carmingnac, Lund (SE)

(72) Inventors: Madeleine Durbeej-Hjalt, Lomma (SE); Virginie Carmingnac, Lund (SE)

(73) Assignee: MD PHARMA AB, Lund (SE)

(21) Appl. No.: 14/122,227

(22) PCT Filed: May 27, 2011
(86) PCT No.: PCT/GB2011/000817

§ 371 Date: Mar. 26, 2014

(51) Int. Cl. A61K 31/52

(52) U.S. Cl. CPC A61K 31/52
USPC 514/171; 514/263.4

(57) ABSTRACT

The present invention provides an inhibitor of intracellular protein degradation for use in the treatment and prevention of muscular dystrophy in a mammal. In particular, the invention provides an autophagy inhibitor and/or an inhibitor of the ubiquitin-proteasome system (such as a proteasome inhibitor) for use in the treatment and prevention of muscular dystrophy (such as congenital muscular dystrophy [e.g. MDC1A) and Duchenne muscular dystrophy [DMD]). The invention further provides corresponding methods of treatment and prevention of muscular dystrophy.

### FIELD OF INVENTION

The present invention relates to agents and methods for the treatment and prevention of muscular dystrophy. In particular, the invention provides inhibitors of intracellular protein degradation (such as autophagy inhibitors and/or proteosome inhibitors) for use in the treatment and prevention of muscular dystrophies, including but not limited to laminin-α2-deficient congenital muscular dystrophy and Duchenne muscular dystrophy.

### BACKGROUND

Muscular dystrophy (MD) refers to a group of hereditary muscle diseases that weakens the muscles that move the human body. MDs are characterised by progressive skeletal muscle weakness, defects in muscle proteins, and the death of muscle cells and tissue.

Nine diseases including Duchenne, Becker, limb girdle, congenital, facioscapulohumeral, myotonic, oculopharyngeal, distal, and Emery-Dreifuss are always classified as MD but there are more than one hundred diseases in total with similarities to MD.

Most types of MD are multi-system disorders with manifestations in body systems including the heart, gastrointestinal and nervous systems, endocrine glands, skin, eyes and even brain. The condition may also lead to mood swings and learning difficulties.

There is no specific treatment for any of the forms of MD. MD may lead to a decline in lung function and therefore assisted ventilation may confer significant clinical benefits in MD patients. Physical therapy to prevent contractures and maintain muscle tone, orthoses (orthopedic appliances used for support) and corrective orthopedic surgery may be needed to improve the quality of life in some cases. The cardiac problems that occur with Emery-Dreifuss muscular dystrophy and myotonic muscular dystrophy may require a pacemaker. The myotonia (delayed relaxation of a muscle after a strong contraction) occurring in myotonic muscular dystrophy may be treated with medications such as quinine, phenyloin, or mexiletine, but no actual long term treatment has been found.

Occupational therapy assists the individual with MD in engaging in his/her activities of daily living (self-feeding, self-care activities, etc.) and leisure activities at the most independent level possible. This may be achieved with use of adaptive equipment or the use of energy conservation techniques. Occupational therapy may implement changes to a person's environment, both at home or work, to increase the individual's function and accessibility. Occupational therapists also address psychosocial changes and cognitive decline which may accompany MD, as well as provide support and education about the disease to the family and individual.

New gene-based therapies for MD are emerging with particular noted advances in using conventional gene replacement strategies, RNA-based technology and pharmacological approaches. However, while the proof of principle has been demonstrated in animal models, success in clinical trials has yet to be demonstrated.

Hence, there exists a need for effective agents for the treatment and prevention of MD.

### SUMMARY OF INVENTION

The first aspect of the invention provides an inhibitor of intracellular protein degradation for use in the treatment or prevention of muscular dystrophy in a mammal.

By “inhibitor of intracellular protein degradation” we include any agent (e.g. chemical entity, polypeptide or otherwise) which is capable of inhibiting, at least in part, the endogenous protein degradation pathway(s) in mammalian cells.

Two main pathways are responsible for the degradation of proteins in mammalian cells, the autophagy-lysosome degradation pathway and the ubiquitin-proteosome pathway (for example, see Knecht et al., 2009, Cell Mol Life Sci. 66(15):2427-43 and Sandri, 2010, FEBS Lett. 584(7):1411-6, the disclosures of which are incorporated herein by reference).

In one embodiment, the inhibitor of cellular protein degradation is an autophagy inhibitor.

By “autophagy inhibitor” we include any agent (e.g. small chemical entities, polypeptides [including antibodies] and the like) which is capable of inhibiting, at least in part, the autophagy-lysosome pathway in mammals. It will be appreciated that the agent may inhibit such autophagocytosis either directly (by acting on a component of the autophagy-lysosome pathway) or indirectly (by acting on another cell component or factor that itself inhibits, directly or indirectly, the autophagy-lysosome pathway).

Regulation of the autophagy-lysosome pathway in mammals is discussed in detail in the scientific literature (for example, see Mehrpour et al., 2010, Cell Res. 20(7):748-62 and Mehrpour et al., 2010, Am J Physiol Cell Physiol. 298(4):C776-85, the disclosures of which are incorporated herein by reference).

Examples of autophagy inhibitors are well-known in the art, in part through their suggest use in the treatment of cancer (for example, see Livesey et al, 2009, Curr Opin Investig Drugs. 10(12):1269-79, the disclosures of which are incorporated herein by reference).

It will be appreciated by skilled persons that the autophagy inhibitor may be capable of inhibiting, in whole or in part, macroautophagy, microautophagy and/or chaperone-mediated autophagy.

In one embodiment, the autophagy inhibitor is a macroautophagy inhibitor.

Thus, the autophagy inhibitor may be selected from the group consisting of 3-methyladenine, wortmannin, bafilomycins (such as bafilomycin A1), chloroquine, hydroxychloroquine, PI3K class III inhibitors (such as LY294002), L-asparagine, catalase, E64, leupeptin, N-acetyl-L-cysteine, pepstatin A, propylamine, 4-aminoquionolines, 3-methyl adenosine, adenosine, okadaic acid, N6-mercaptopurine riboside (N-6-MPR), an aminothiolated adenosine analogue and 5-amino-4-imidazole carboxamide riboside (AICAR).

In one embodiment, the inhibitor of cellular protein degradation is an inhibitor of the ubiquitin-proteasome system.

By “inhibitor of the ubiquitin-proteasome system” we mean an agent (e.g. small chemical entity, polypeptide or the like) which is capable of inhibiting, at least in part, a function of the ubiquitin-proteasome system (preferably in vivo in humans). Such an inhibitor may act at any point along the ubiquitin-proteasome protein degradation pathway, for example by inhibiting (at least, in part) the marking of proteins for degradation by modulating ubiquitination or deubiquitination, by inhibiting the ability of the proteasome to recognize or bind proteins to be degraded, and/or by inhibiting the ability of the proteasome to degrade proteins.

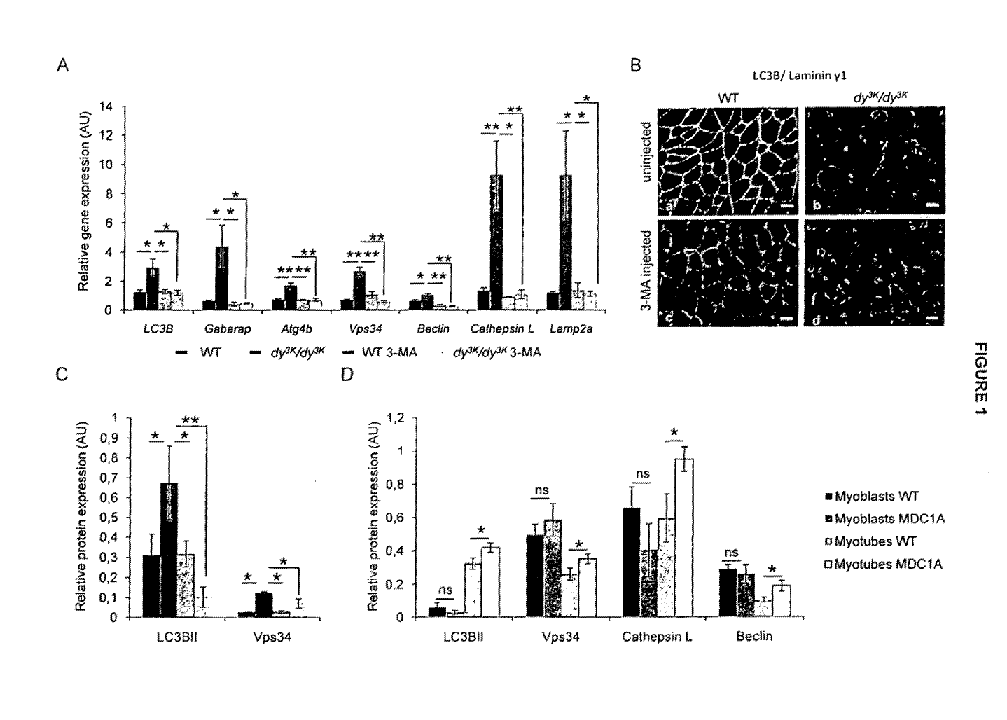

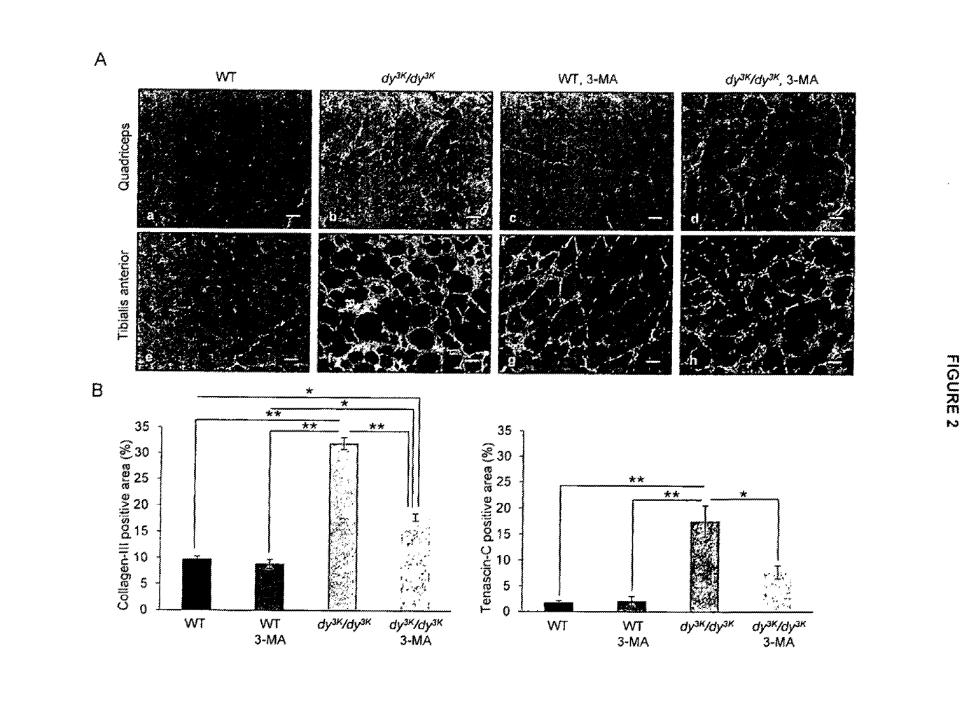

The ubiquitin-proteasome system, and components thereof, are described in detail in the scientific literature, for example see Ciechanover, 1998, The EMBO Journal 17, 7151-7160 (see FIGS. 1 and 2 therein) and Bedford et al., 2011, Nat Rev Drug Discov 10, 29-46; the disclosures of which are incorporated herein by reference.

In one embodiment, the inhibitor of the ubiquitin-proteasome system is a proteasome inhibitor acting directly upon the proteasome to inhibit its function. For example, the proteasome inhibitor may inhibit (at least, in part) the ability of the human proteasome to degrade proteins. Examples of proteasome inhibitors are well known in the art (for example, see de Bettignies & Coux, 2010, Biochimie. 92(11):1530-45, Kling et al., 2010, Nature Biotechnology, 28(12):1236-1238, the disclosures of which are incorporated herein by reference).

Thus, the inhibitor of the ubiquitin-proteasome system may be a proteasome inhibitor selected from the group consisting of bortezomib (PS-341, MG-341, Velcade®), PI-083, MLN 9708, MLN 4924, MLN 519, carfilzomib, ONX 0912, CEP-1877, NPI-0047, NPI-0052, BU-32 (NSC D750499-S), PR-171, IPSI-001, disulfuram, epigallocatechin-3-gallate, MG-132, MG-262, salinosporamide A, leupeptin, calpain inhibitor I, calpain inhibitor II, MG-115, PSI (Z-Ile-Glu(OtBu)-Ala-Leu-H (aldehyde)), peptide glyoxal, peptide alpha-ketoamide, peptide boronic ester, peptide benzamide, P′-extended peptide alpha-ketoamide, lactacystin, clastro-lactacystin β [symbol] , lactone, epoxomicin, eponemycin, TCM-86A, TCM-86B, TCM 89, TCM-96, YU101, TCM-95, gliotoxin, the T-L activity specific aldehyde developed by Loidl et al., (Chem. Biol., (1999) 6:197-204), HNE (4-hydroxy-2-nonenal), YU102 and natural products with proteasome-inhibitory effects, such as green tea polyphenol (−)-epigallocatechin-3-gallate (EGCG), soy isoflavone genistein, and the spice turmeric compound curcumin.

For example, the proteasome inhibitor may be bortezomib (Proprietary name=Velcade®, IUPAC name=[(1R)-3-methyl-({(2S)-3-phenyl-2-[(pyrazin-2-ylcarbonyl)-amino]propanoyl}amino)butyl]boronic acid, CAS number=179324-69-7).

It will be further appreciated by persons skilled in the art that the present invention provides agents suitable for the treatment and prevention of several different forms of muscular dystrophy and muscular dystrophy-like indications, such as related myopathies.

In one embodiment, the muscular dystrophy is selected from the group consisting of congenital muscular dystrophy, Duchenne muscular dystrophy (DMD), Becker's muscular dystrophy (BMD, Benign pseudohypertrophic muscular dystrophy), distal muscular dystrophy (distal myopathy), Emery-Dreifuss muscular dystrophy (EDMD), facioscapulohumeral muscular dystrophy (FSHMD, FSHD or FSH), limb-girdle muscular dystrophy (LGMD), myotonic muscular dystrophy, centronuclear myopathies and oculopharyngeal muscular dystrophy.

Thus, the muscular dystrophy may be a congenital muscular dystrophy, for example selected from the group consisting of
(a) Congenital muscular dystrophy with abnormalities in the extracellular matrix, such as Merosin (laminin α2) deficient CMD (MDC1A) and Collagen VI deficient CMD (Ullrich CMD and Bethlem myopathy);
(b) Dystroglycanopathies (abnormalities of α-dystroglycan), such as Fukuyama-type CMD, Variants of muscle-eye brain disease, Walker-Warburg syndrome, Congenital muscular dystrophy type IC, Congenital muscular dystrophy type 1D and Limb-girdle muscular dystrophy 21;
(c) Defects in the integrin α7 subunit, such as Congenital myopathy with integrin α7 deficiency;
(d) Abnormalities of nuclear envelope proteins, such as L-CMD;
(e) Abnormalities in ER, such as SEPN1 related myopathy (formerly known as Rigid Spine Muscular Dystrophy);
(f) Undiagnosed CMD, including merosin positive; and
(g) Ryanodine receptor gene (RYR1) CMD

In one preferred embodiment, the muscular dystrophy is laminin-α2-deficient congenital muscular dystrophy (Muscular Dystrophy, Congenital Merosin-Deficient, 1a/MDC1A).

However, in another embodiment the muscular dystrophy is not laminin-α2-deficient congenital muscular dystrophy (Muscular Dystrophy, Congenital Merosin-Deficient, 1a/MDC1A).

In an alternative embodiment, the muscular dystrophy is the muscular dystrophy is Duchenne muscular dystrophy (DMD).

In a further embodiment, the muscular dystrophy is a distal muscular dystrophy (distal myopathy), for example selected from the group consisting of Miyoshi myopathy, distal myopathy with anterior tibial onset, and Welander distal myopathy.

In a further embodiment, the muscular dystrophy is an Emery-Dreifuss muscular dystrophy (EDMD), for example selected from the group consisting of EDMD1, EDMD2, EDMD3, EDMD4, EDMD5 and EDMD6.

In a further embodiment, the muscular dystrophy is a facioscapulohumeral muscular dystrophy (FSHMD, FSHD or FSH), for example selected from the group consisting of FSHMD1A (4q35 deletion) and FSHMD1B.

In a further embodiment, the muscular dystrophy is a Limb-girdle muscular dystrophy or (Erb's muscular dystrophy), for example selected from the group consisting of LGMD1A, LGMD1B, LGMD1C, LGMD1D, LGMD1E, LGMD1F, LGMD1G, LGMD2A, LGMD2B, LGMD2C, LGMD2D, LGMD2E, LGMD2F, LGMD2G, LGMD2H, LGMD2I, LGMD2J, LGMD2K, LGMD2L, LGMD2M, LGMD2N and LGMD20.

In a still further embodiment, the muscular dystrophy is a myotonic dystrophy, for example selected from the group consisting of DM1 (also called Steinert's disease) severe congenital form, DM1 childhood-onset form and DM2 (also called proximal myotonic myopathy or PROMM).

As discussed above, the term “muscular dystrophy” encompasses a number of related hereditary diseases associated with weakening of the muscles that move the body.

In one embodiment, the muscular dystrophy is associated with excessive autophagy (i.e. excessive macroautophagy, microautophagy and/or chaperone-associated autophagy).

Thus, the muscular dystrophy may be associated with excessive macroautophagy. For example, the muscular dystrophy may be laminin-α2-deficient congenital muscular dystrophy, MDC1A).

In an alternative embodiment, the muscular dystrophy is not associated with macroautophagy dysregulation. For example, the muscular dystrophy may be Duchenne muscular dystrophy).

In a further alternative embodiment, the muscular dystrophy is not associated with reduced macroautophagy.

The inhibitors of intracellular protein degradation of the invention are for use in the treatment and/or prevention of muscular dystrophy.

By “treatment and/or prevention” we mean that the inhibitor of intracellular protein degradation is used to prevent, reduce and/or eliminate one or more symptoms or parameters associated with muscular dystrophy.

In one embodiment, the treatment or prevention of muscular dystrophy results in one or more of the following parameters being reduced in the mammal:
(i) muscle fibrosis;
(ii) muscle atrophy;
(iii) muscular apoptosis (caspase-3 positive muscle fibres);
(iv) collagen II expression;
(v) tenascin-C expression,
(vi) proportion of muscle fibre cells with centrally locate nuclei; and/or
(vii) laminin alpha-4 expression.

Methods for the assessment of these parameters are well known in the art (for example, see Gawlik et al, 2010, PLoS ONE 5(7):e11549 and Meinen et al., 2007, J Cell Biol. 176(7):979-93).

Said reduction in the parameter(s) may be in whole or in part. For example, the one or more parameters may be reduced by at least 10%, for example, by at least 15%, 20%, 25%, 30%, 35%, 40%, 45%, 50%, 60%, 70%, 80%, 90% or by 100% relative to the level prior to treatment with the inhibitor.

Alternatively, or additionally, the treatment or prevention of muscular dystrophy may result in one or more of the following parameters being increased in the mammal:
(a) muscle regeneration;
(b) muscle weight;
(c) average muscle fibre diameter;
(d) ratio of quadriceps muscle wet weight per body weight
(e) lifespan;
(f) locomotive function;
(g) laminin beta-2 expression;
(h) proportion of muscle fibre cells with centrally locate nuclei;
(i) expression of MyoD1 in satellite cells; and/or
(j) expression of eMHC in regenerating muscle fibres.

Methods for the assessment of these parameters are well known in the art (for example, see Gawlik et al., 2010, PLoS ONE 5(7):e11549 and Meinen et al., 2007, J. Cell Biol. 176(7):979-93, the disclosures of which are incorporated herein by reference).

Said increase in the parameter(s) may be in whole or in part. For example, the one or more parameters may be increased by at least 10%, for example, by at least 15%, 20%, 25%, 30%, 35%, 40%, 45%, 50%, 60%, 70%, 80%, 90%, 100%, 125%, 150%, 175%, 200%, 250%, 300%, 350%, 450%, 500%, 600%, 700%, 800%, 900% or 1000% relative to the level prior to treatment with the inhibitor.

Alternatively, or additionally, the treatment or prevention of muscular dystrophy may result in Akt phosphorylation at threonine 308 and/or 473 being restored to wild type or near wild type levels, for example, within 35%, 30%, 25%, 20%, 15%, 14%, 13%, 12%, 11%, 10%, 9%, 8%, 7%, 6%, 5%, 4%, 3%, 2%, 1%, 0.5%, 0.25%, 0.1%, 0.05% of wild type levels.

It will be appreciated by persons skilled in the art that the inhibitors of intracellular protein degradation of the invention may be for use in combination with a second therapeutic agent or treatment for muscular dystrophy.

For example, the second therapeutic agent or treatment may comprise or consist of physical therapy, corrective orthopedic surgery and/or steroids.

Alternatively, or in addition, the second therapeutic agent or treatment may comprise or consist of gene replacement, cell therapy and/or anti-apoptosis therapy (for example, see Gawlik et al., 2004, Hum Mol. Genet. 13(16):1775-84, Hagiwara at al., 2006, FEBS Lett. 580(18):4463-8, Meinen at al, 2007, J. Cell Biol. 176(7):979-93 and Girgenrath et al., 2009, Ann Neurol. 65(1) 47-56, the disclosures of which are incorporated herein by reference).

In one embodiment, the inhibitor of intracetylular protein degradation is an autophagy inhibitor for use in combination with a proteasome inhibitor, or vice-versa. Such combination therapies thus seek to inhibit both of the main pathways of protein degradation in mammalian cells.

It will be appreciated by persons skilled in the art that the inhibitors of the invention may be for use in any mammal.

In one embodiment, the mammal is a human.

Alternatively, the mammal may be a dog, cat, horse, or other domestic or farm mammalian animal.

A second aspect of the invention provides the use of an inhibitor of intracellular protein degradation in the preparation of a medicament for the treatment or prevention of muscular dystrophy in a mammal.

Examples of suitable inhibitors of intracellular protein degradation are disclosed above in relation to the first aspect of the invention.

Thus, in one embodiment, the inhibitor of cellular protein degradation is an autophagy inhibitor. For example, the autophagy inhibitor may be selected from the group consisting of 3-methyladenine, wortmannin, bafilomycins (such as bafilomycin A1), chloroquine, hydroxychloroquine, PI3K class III inhibitors (such as LY294002), L-asparagine, catalase, E64D, leupeptin, N-acetyl-L-cysteine, pepstatin A, propylamine, 4-aminoquionolines, 3-methyl adenosine, adenosine, okadaic acid, N⁶-mercaptopurine riboside (N-6-MPR), an aminothiolated adenosine analogue and 5-amino-4-imidazole arboxamide riboside (AICAR).

In an alternative embodiment, the inhibitor of cellular protein degradation is an inhibitor of the ubiquitin-proteasome system.

Thus, in one embodiment, the inhibitor of cellular protein degradation is a proteasome inhibitor. For example, the proteasome inhibitor may be selected from the group consisting of bortezomib (PS-341, MG-341, Velcade®), PI-083, MLN 9708, MLN 4924, MLN 519, carfilzomib, ONX 0912, CEP-1877, NPI-0047, NPI-0052, BU-32 (NSC D750499-S), PR-171, IPSI-001, disulfuram, epigallocatechin-3-gallate, MG-132, MG-262, salinosporamide A, leupeptin, calpain inhibitor I, calpain inhibitor II, MG-115, PSI (Z-Ile-Glu(OtBu)-Ala-Leu-H (aldehyde)), peptide glyoxal, peptide alpha-ketoamide, peptide boronic ester, peptide benzamide, P′-extended peptide alpha-ketoamide, lactacystin, clastro-lactacystin P3′-lactone, epoxomicin, eponemycin, TCM-86A, TCM-86B, TCM 89, TCM-96, YU101, TCM-95, gliotoxin, the T-L activity specific aldehyde developed by Loidl et al., (Chem. Biol., (1999) 6:197-204), HNE (4-hydroxy-2-nonenal), YU102 and natural products with proteasome-inhibitory effects, such as green tea polyphenol (−)-epigallocatechin-3-gallate (EGCG), soy isoflavone genistein, and the spice turmeric compound curcumin.

The uses of the second aspect of the invention extend to the same muscular dystrophy indications disclosed above in relation to the first aspect of the invention.

Thus, in one embodiment, the muscular dystrophy is selected from the group consisting of congenital muscular dystrophy, Duchenne muscular dystrophy (DMD), Becker's muscular dystrophy (BMD, Benign pseudohypertrophic muscular dystrophy), distal muscular dystrophy (distal myopathy), Emery-Dreifuss muscular dystrophy (EDMD), facioscapulohumeral muscular dystrophy (FSHMD, FSHD or FSH), limb-girdle muscular dystrophy (LGMD), myotonic muscular dystrophy and oculopharyngeal muscular dystrophy.

For example, the muscular dystrophy may be laminin-α2-deficient congenital muscular dystrophy (Muscular Dystrophy, Congenital Merosin-Deficient, 1a/MDC1A).

In an alternative embodiment, the muscular dystrophy is not laminin-α2-deficient congenital muscular dystrophy (Muscular Dystrophy, Congenital Merosin-Deficient, 1a/MDC1A).

In a further alternative embodiment, the muscular dystrophy is Duchenne muscular dystrophy (DMD).

It will be appreciated by persons skilled in the art that the uses of the second aspect of the invention may provide medicaments for use in any mammal (see above).

In one embodiment, the mammal is a human.

In relation to all aspects of the invention, the inhibitors of intracellular protein degradation may be formulated at various concentrations, depending on a number of factors including the efficacy/toxicity of the inhibitor being used and the indication for which it is being used. Of course, the maximum concentration in any given pharmaceutical formulation will be limited by the maximum solubility of the inhibitor therein. However, the formulations should contain an amount of the inhibitor sufficient to provide an in vivo concentration sufficient to inhibit, at least in part, intracellular degradation of proteins in muscle cells and other cell types (e.g. Schwann cells) affected by the disease.

In one embodiment, the inhibitor of intracellular protein degradation is formulated at a concentration of between 1 nM and 1M. For example, the pharmaceutical formulation may comprise the inhibitor at a concentration of between 1 μM and 1 mM, for example between 1 μM and 100 μM, between 5 μM and 50 μM, between 10 μM and 50 μM, between 20 μM and 40 μM or about 30 μM. The inhibitors of intracellular protein degradation will generally be administered in admixture with a suitable pharmaceutical excipient, diluent or carrier selected with regard to the intended route of administration and standard pharmaceutical practice (for example, see Remington: The Science and Practice of Pharmacy, 19^{th} edition, 1995, Ed. Alfonso Gennaro, Mack Publishing Company, Pennsylvania, USA, the disclosures of which are incorporated herein by reference). Suitable routes of administration are discussed below, and include intravenous, oral, pulmonary, intranasal, topical, aural, ocular, bladder and CNS delivery.

For example, the inhibitor of intracellular protein degradation may be administered orally, buccally or sublingually in the form of tablets, capsules, ovules, elixirs, solutions or suspensions, which may contain flavouring or colouring agents, for immediate-, delayed- or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The formulations may alternatively be administered parenterally, for example, intravenously, intraarterially, intratumorally, peritumorally, intraperitoneally, intrathecally, intraventricularly, intrasternally, intracranially, intra-muscularly or subcutaneously (including via an array of fine needles or using needle-free Powderject® technology), or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

The inhibitors of intracellular protein degradation may also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetra-fluoroethane, a hydrofluoroalkane such as 1,1,1,2-tetrafluoroethane (HFA 134A³ or 1,1,1,2,3,3,3-heptafluoropropane (HFA 227EA³), carbon dioxide or other suitable gas. In the case of a pressurised aerosol, the dosage unit may be determined by providing a valve to deliver a metered amount. The pressurised container, pump, spray or nebuliser may contain a solution or suspension of the active inhibitor, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains at least 1 mg of a compound for delivery to the patient. It will be appreciated that the overall dose with an aerosol will vary from patient to patient and from indication to indication, and may be administered in a single dose or, more usually, in divided doses throughout the day.

Alternatively, other conventional administration routes known in the art may also be employed; for example the formulation of the invention may be delivered orally, buccally or sublingually in the form of tablets, capsules, ovules, elixirs, solutions or suspensions, which may contain flavouring or colouring agents, for immediate-, delayed- or controlled-release applications. The formulation may also be administered intra-ocularly, intra-aurally or via intracavemosal injection (see below).

For application topically, e.g. to the skin, the inhibitor of intracellular protein degradation can be administered in the form of a lotion, solution, cream, gel, ointment or dusting powder (for example, see Remington, supra, pages 1586 to 1597). Thus, the inhibitors can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, e-lauryl sulphate, an alcohol (e.g. ethanol, cetearyl alcohol, 2-octyldodecanol, benzyl alcohol) and water.

Formulations suitable for topical administration in the mouth further include lozenges comprising the active ingredient in a flavoured basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

The formulation may also be administered by the ocular route, particularly for treating diseases of the eye. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For veterinary use, a compound is administered as a suitably acceptable formulation in accordance with normal veterinary practice and the veterinary surgeon will determine the dosing regimen and route of administration which will be most appropriate for a particular animal.

In one preferred embodiment, the formulation is suitable for systemic administration to a patient (for example, via an oral or parenteral administration route).

The formulation comprising the inhibitor of intracellular protein degradation may be stored in any suitable container or vessel known in the art. It will be appreciated by persons skilled in the art that the container or vessel should preferably be airtight and/or sterilised. Advantageously, the container or vessel is made of a plastics material, such as polyethylene.

A third, related aspect of the invention provides a method for treating or preventing muscular dystrophy in a mammal comprising administering an effective amount of an inhibitor of intracellular protein degradation to the mammal.

Examples of suitable inhibitors of intracellular protein degradation are disclosed above in relation to the first aspect of the invention.

Thus, in one embodiment, the inhibitor of cellular protein degradation is an autophagy inhibitor. For example, the autophagy inhibitor may be selected from the group consisting of 3-methyladenine, wortmannin, bafilomycins (such as bafilomycin A1), chloroquine, hydroxychloroquine, PI3K class III inhibitors (such as LY294002), L-asparagine, catalase, E640, leupeptin, N-acetyl-L-cysteine, pepstatin A, propylamine, 4-aminoquionolines, 3-methyl adenosine, adenosine, okadaic acid, N6-mercaptopurine riboside (N-6-MPR), an aminothiolated adenosine analogue and 5-amino-4-imidazole carboxamide riboside (AICAR).

In an alternative embodiment, the inhibitor of cellular protein degradation is an inhibitor of the ubiquitin-proteasome system.

Thus, the inhibitor of cellular protein degradation may be a proteasome inhibitor. For example, the proteasome inhibitor may be selected from the group consisting of bortezomib (PS-341, MG-341, Velcade®), PI-083, MLN 9708, MLN 4924, MLN 519, carfilzomib, ONX 0912, CEP-1877, NPI-0047, NPI-0052, BU-32 (NSC D750499-S), PR-171, IPSI-001, disulfuram, epigallocatechin-3-gallate, MG-132, MG-262, salinosporamide A, leupeptin, calpain inhibitor I, calpain inhibitor II, MG-115, PSI (Z-Ile-Glu(OtBu)-Ala-Leu-H (aldehyde)), peptide glyoxal, peptide alpha-ketoamide, peptide boronic ester, peptide benzamide, P′-extended peptide alpha-ketoamide, lactacystin, clastro-lactacystin β [symbol] -lactone, epoxomicin, eponemycin, TCM-86A, TCM-86B, TCM 89, TCM-96, YU101, TCM-95, gliotoxin, the T-L activity specific aldehyde developed by Loidi et al., (Chem. Biol., (1999) 6:197-204), HNE (4-hydroxy-2-nonenal), YU102 and natural products with proteasome-inhibitory effects, such as green tea polyphenol (−)-epigallocatechin-3-gallate (EGCG), soy isoflavone genistein, and the spice turmeric compound curcumin.

The methods of the third aspect of the invention extend to the same muscular dystrophy indications disclosed above in relation to the first aspect of the invention.

Thus, in one embodiment, the muscular dystrophy is selected from the group consisting of congenital muscular dystrophy, Duchenne muscular dystrophy (DMD), Becker's muscular dystrophy (BMD, Benign pseudohypertrophic muscular dystrophy), distal muscular dystrophy (distal myopathy), Emery-Dreifuss muscular dystrophy (EDMD), facioscapulohumeral muscular dystrophy (FSHMD, FSHD or FSH), limb-girdle muscular dystrophy (LGMD), myotonic muscular dystrophy and oculopharyngeal muscular dystrophy.

For example, the muscular dystrophy may be laminin-α2-deficient congenital muscular dystrophy (Muscular Dystrophy, Congenital Merosin-Deficient, 1a/MDCIA).

In an alternative embodiment, the muscular dystrophy is not laminin-α2-deficient congenital muscular dystrophy (Muscular Dystrophy, Congenital Merosin-Deficient, 1a/MDC1A).

In a further alternative embodiment, the muscular dystrophy is Duchenne muscular dystrophy (DMD).

It will be appreciated by persons skilled in the art that the methods of the third aspect of the invention may be performed on any mammal (see above).

In one embodiment, the mammal is a human.

In the methods and uses of the invention, the inhibitor of intracellular protein degradation will be administered to a patient in a pharmaceutically effective dose. A ‘therapeutically effective amount’, or ‘effective amount’, or ‘therapeutically effective’, as used herein, refers to that amount which provides a therapeutic effect for a given muscular dystrophy indication and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. The administration of the pharmaceutically effective dose can be carried out both by single administration in the form of an individual dose unit or else several smaller dose units and also by multiple administrations of subdivided doses at specific intervals.

Alternatively, the dose may be provided as a continuous infusion over a prolonged period.

In one embodiment, the inhibitor of intracellular protein degradation is for administration at a dose sufficient to inhibit, at least in part, protein degradation in muscle cells in the patient being treated. Thus, the dose of the inhibitor may be chosen in order to inhibit protein degradation in muscle cells in the patient.

Typically, the dose of the inhibitor of intracellular protein degradation will be within the range of 0.01 to 100 mg/kg per administration (e.g. daily; see below), for example between 0.05 and 50 mg/kg, 0.1 and 20 mg/kg, 0.01 and 10 mg/kg, 0.1 and 5.0 mg/kg, 0.5 and 3.0 mg/kg or between 1 and 1.5 mg/kg per administration.

It will be appreciated that the dose of inhibitor of intracellular protein degradation may be changed during the course of treatment of the patient. For example, a higher dose may be used during an initial therapeutic treatment phase, followed by a lower ‘maintenance’ dose after the initial treatment is complete to prevent recurrence of the condition.

In one embodiment, the inhibitor of intracellular protein degradation is administered systemically.

In another embodiment, the inhibitor of intracellular protein degradation is orally.

In a further embodiment, the inhibitor of intracellular protein degradation is administered repeatedly, for example every 4 hours, 6 hours, 12 hours, 24 hours, 48 hours, twice weekly, weekly, twice monthly, or monthly.

It will be appreciated by persons skilled in the art that the inhibitor of intracellular protein degradation may be administered as a sole treatment for muscular dystrophy in a patient or as part of a combination treatment with one or more further therapeutic agents or treatments.

In one embodiment, the further therapeutic agent or treatment comprises physical therapy, corrective orthopedic surgery and/or steroids.

Alternatively, or in addition, the second therapeutic agent or treatment may comprise or consist of gene replacement, cell therapy and/or anti-apoptosis therapy.

In a further embodiment of the third aspect of the invention, the method comprises administering an autophagy inhibitor in combination with an inhibitor of the ubiquitin-proteasome system. For example, an inhibitor of macroautophagy may be administered to the patient in combination with a proteasome inhibitor.

Preferred, non-limiting examples which embody certain aspects of the invention will now be described, with reference to the following figures:

FIG. 1: Autophagy is increased in skeletal muscle from dy3K/dy3K mice. (A) Relative amounts of LC3B, Gabarapl1, Atg4b, Vps34, Beclin, Cathepsin L and Lamp2a mRNAs in 3.5-week-old wild-type, dy3KIdy3K mice and in 4.5-week-old 3-MA injected wild-type and dy3K/dy3K mice (n=6 for each group). The GAPDH gene expression served as a reference. (B) Left panel: Co-immunostaining on cross sections of quadriceps muscles from uninjected wild-type (a, n=5) and dy3K/dy3K (b, n=5) mice and 3-MA wild type (c, n=6) and dy3K/dy3K (d and e, n=6) mice. LC3B (in red) is present in autophagosomes and laminin γ1 chain (in green) serves as delineating fiber boundaries. Bar=40 μm. (C) Densitometry analysis of LC3B and Vps34 Western blot analysis in quadriceps muscle from wild-type and dy3K/dy3K mice (3.5-week-old; n=6 per group). Results are expressed in arbitrary units (AU). Labeling of tubulin served as internal loading control. (D) Densitometric analysis of LC3B, Vps34, Cathepsin L and Beclin-1 in human primary myoblasts and myotubes from a control and a laminin α2 chain deficient patient. Data represent mean of 4 different culture wells are expressed in arbitrary units (AU). *, p<0.05; **, p<0.001.

FIG. 2: Muscle morphology is improved and fibrosis is reduced in skeletal muscle with systemic injection of 3-MA. (A) Hematoxylin-eosin staining of cross-sections of quadriceps (a-d) and tibialis anterior (e-h) muscles from wild-type (a, e), non-injected dy3K/dy3K (b, f), injected wild-type and dy3K/dy3K at 2.5 and 3.5 weeks of age (c, d and g, h respectively). Fourteen days later, muscles were isolated and stained. (B) Densitometric quantification of fibrosis in quadriceps muscles from 3-MA wild-type and dy3K/dy3K injected mice (n=6 for each genotype) or non-injected dy3K/dy3K (n=7) and wild-type mice (n=9). Left panel: Percentage of collagen III positive labeling of the total area of the section. Right panel: Percentage of tenascin-C positive labeling of the total area of the cross-section. *, p<0.05; **, p<0.001.

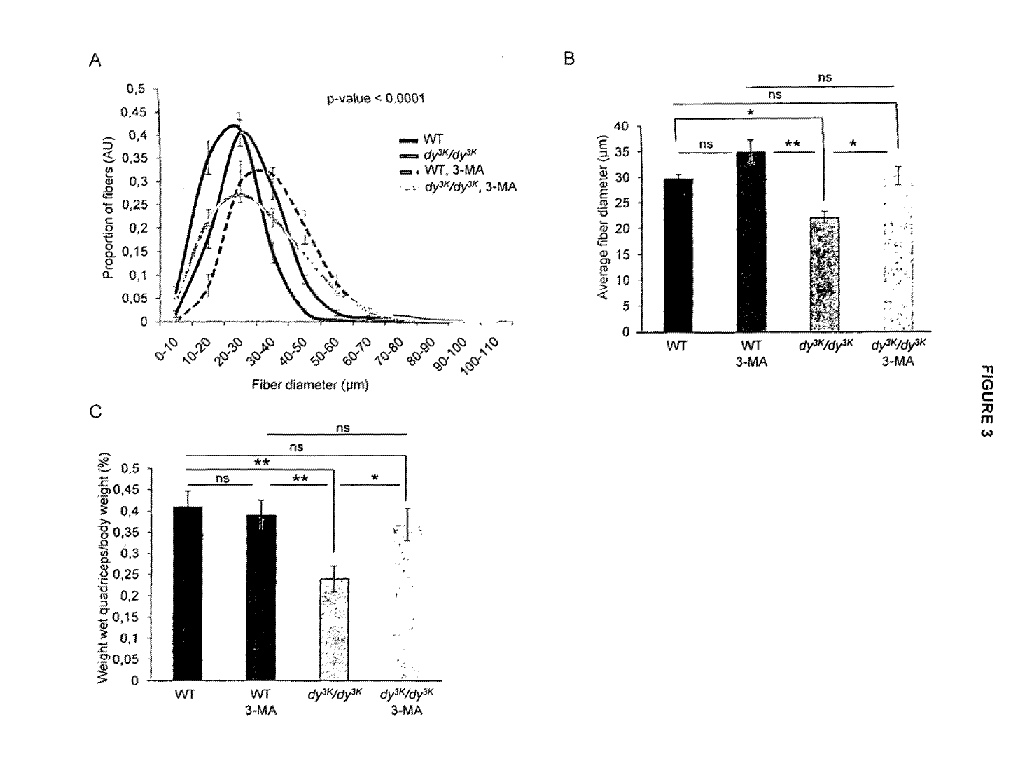

FIG. 3: Atrophy is prevented in quadriceps muscle from 3-MA treated dy3K/dy3K mice. (A) Determination of fiber diameter repartition (in percentage of total number of fibers) in 3-MA injected (green and orange) and non-injected (blue and red) wild-type and dy3K/dy3K mice respectively. A significant difference exists between the curves (p<0.0001) (B) Average of fiber diameter in μm. (C) Proportion (in percentage) of quadriceps muscle wet weight per body weight (wild-type, n=5; dy3K/dy3K, n=4; injected mice, n=5 respectively). *, p<0.05; **, p<0.001.

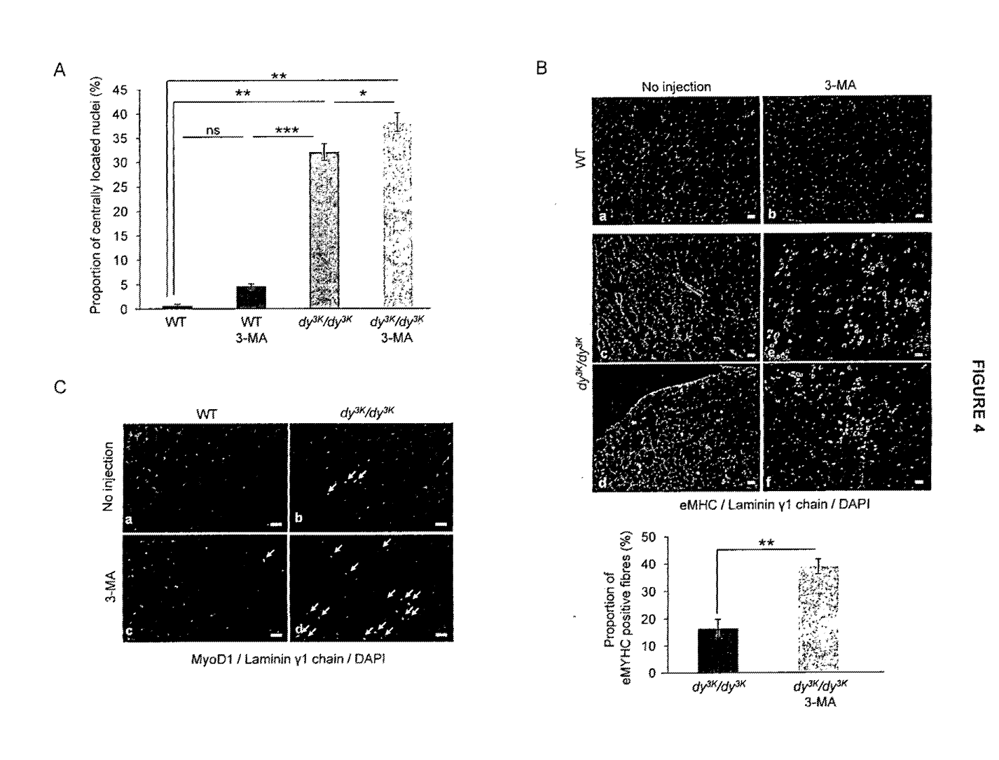

FIG. 4: Systemic injection of 3-MA stimulates regeneration in quadriceps of dy3K/dy3K mice. (A) Proportion of fibers with centrally located nuclei in 3-MA injected dy3K/dy3K and wild-type mice, non-injected dy3K/dy3K and wild-type mice (n=6 for each group). (B) Upper part: Co-immunolabeling on cross sections of quadriceps muscles from uninjected wild-type (n=5) and dy3K/dy3K (n=5) mice and 3-MA injected wild-type (n=6) and dy3K/dy3K (n=6) mice. Laminin γ1 chain (red) delineates fiber boundaries and embryonic myosin heavy chain (eMHC) (green) is expressed only by regenerative fibers. DAPI (in blue) denotes nuclei. Bar=40 μm. Lower part: Percentage of fibers expressing eMHC. (C) Co-immunostaining using antibodies against laminin γ1 chain (red), MyoD (green) and DAPI (blue). Arrows denote MyoD positive nuclei in the interstitial space between myofibers *, p<0.05; *, p<0.001.

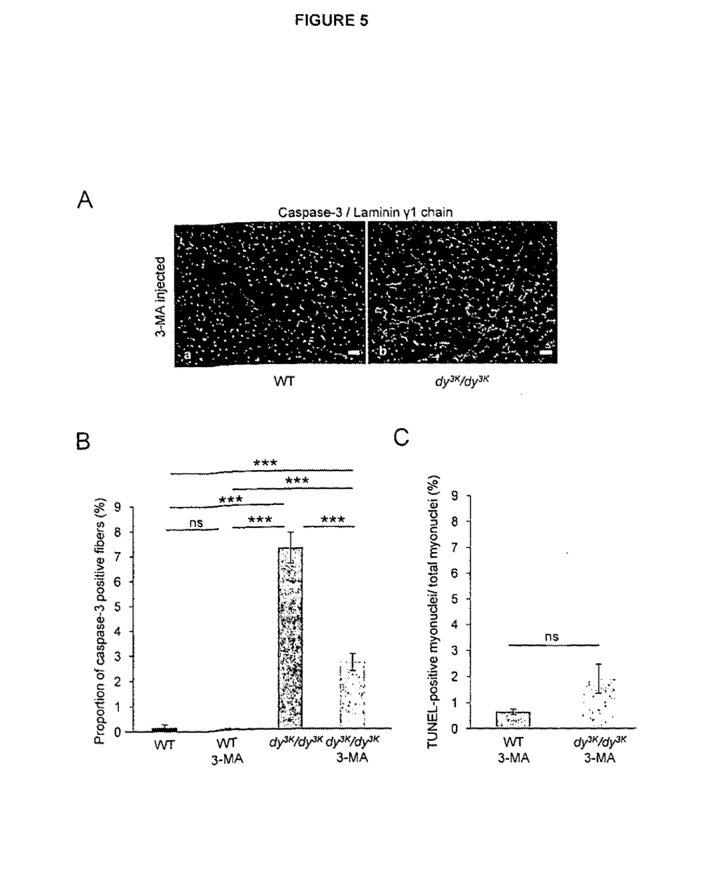

FIG. 5: Apoptosis is diminished after systemic injection of 3-MA in dy3KIdy3K mice. (A) Co-immunostaining using antibodies against pro-caspase 3 and caspase 3 isoforms (green) and laminin γ1 chain (red) in 3-MA injected wild-type and dy3K/dy3K quadriceps muscle (a, b). Fourteen days after 3-MA systemic injection, green positive fibers were found in restricted areas of dy3K/dy3K quadriceps muscle, (b) but most parts of the muscle are marked by the absence of apoptotic fibers (a). Scale bar=40 μm. (B) Percentage of caspase 3 positive fibers in whole quadriceps muscle sections from 3-MA injected dy3K/dy3K and wild-type mice (n=6 for both). (C) Percentage of TUNEL-positive myonuclei in whole quadriceps sections from 3-MA injected dy3K/dy3K and wild-type mice (n=6 for each group). *, p<0.05; ***; p<0.001.

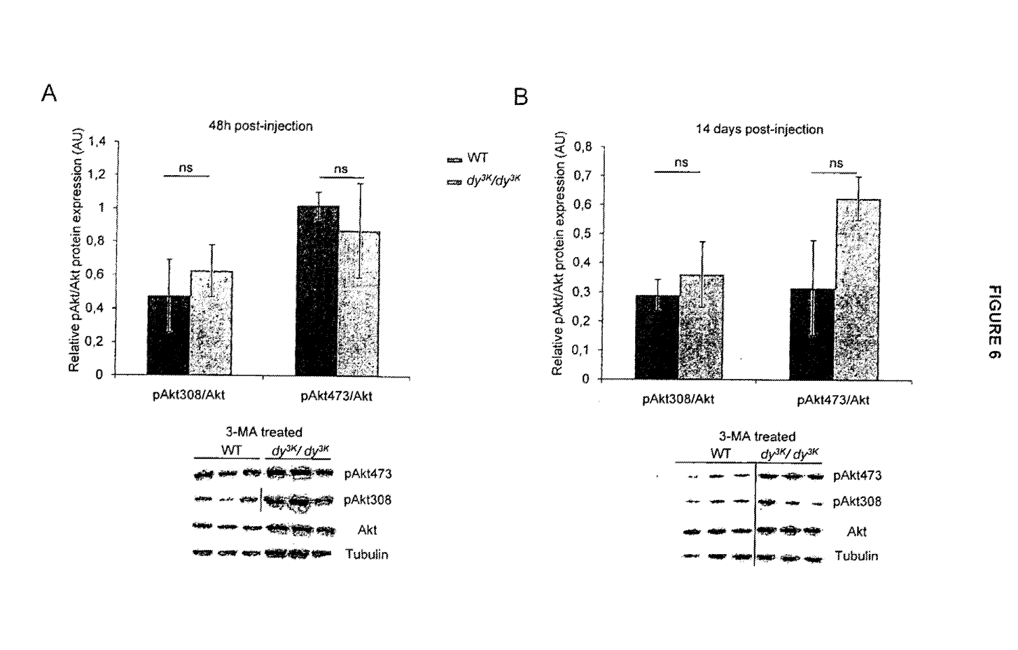

FIG. 6: Akt signaling is restored upon autophagy inhibition. Densitometric analysis and representative Western blot images of phospho-Akt308/Akt and phospho-Akt473/Akt in quadriceps muscle from 3-MA injected wild-type and dy3K/dy3K after 48 hours (A) (n=3 for each genotype) and after 14 days (B) (n=4 and 6 respectively). Data are expressed in arbitrary units (AU) as phospho-Akt is normalized to Akt. Tubulin is used as an internal loading control.

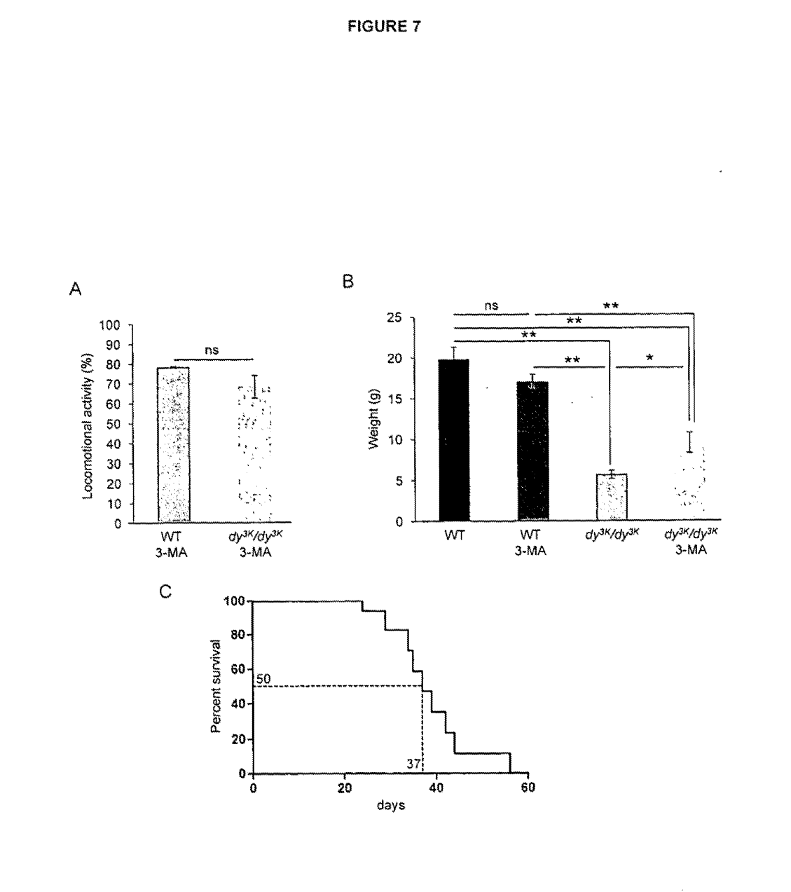

FIG. 7. Systemic injection of 3-MA improves dy3K/dy3K mice locomotion, body weight and survival. (A) Exploratory locomotion of approximately 4-week-old mice in an open field test (n=14 for each group). (B) Body weight measurement of 4-week-old non-injected and 3-MA treated wild type (n=7 respectively) and dy3K/dy3K mice (n=12 respectively) (C) Survival curves of dy3K/dy3K±two systemic injections of 3-MA. The median survival for non-injected dy3K/dy3K mice is 22 days (15) whereas it is 37 days for the treated animals. *p<0.05; **, p<0.001.

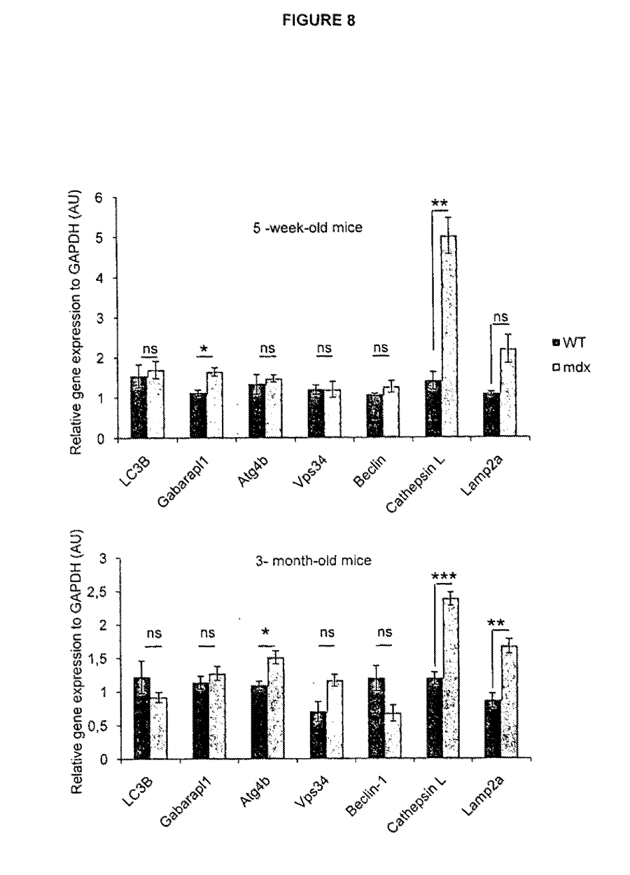

FIG. 8. Autophagy is not upregulated in quadriceps muscle from mdx mice. Relative mRNA expression of LC3B, Cathepsin L, Lamp2a, Gabarapl1, Atg4b, Vps34 and Beclin mRNAs in 5-week-old wild-type and mdx mice (n=6 for each genotype) (upper part) and 3-month-old wild-type and mdx mice (n=3 for each genotype). The GAPDH gene expression served as a reference. *, p<0.01; **, p<0.001; **, p<0.0001.

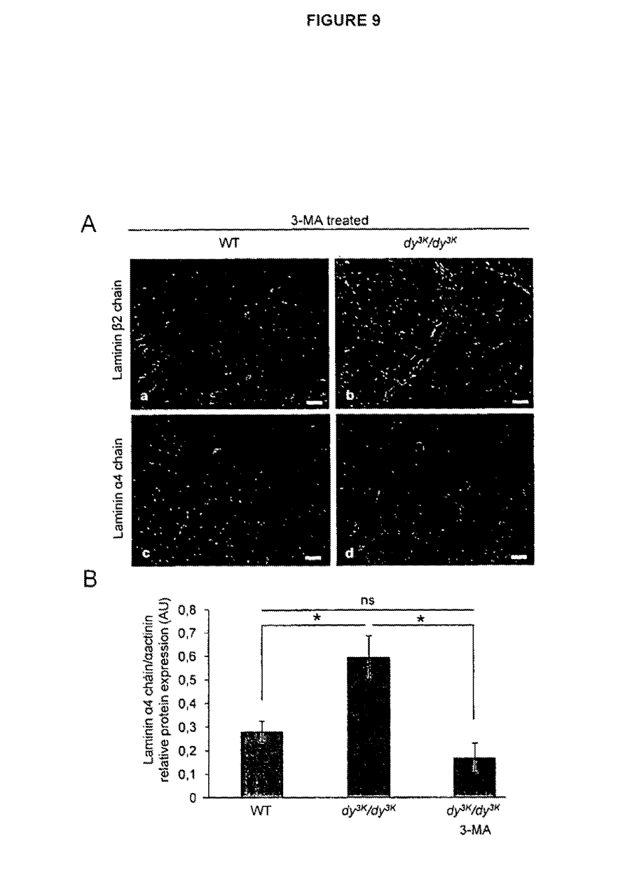

FIG. 9. Systemic injection of 3-MA normalizes laminin α4 and α2 chain expression. (A) Immunofluorescence experiments using antibodies against laminin α2 (a, b) and laminin α4 (c, d) chain on cross-sections of quadriceps muscle from wild-type (a, c) and dyK/de mice (b, d) treated with systemic injection of 3-MA (n=6 for each genotype). Scale bar=40 μm. (B) Densitometric analysis of laminin α4 chain in quadriceps muscle from wild-type, non-treated dy^{3K}/dy^{3K} and 3-MA injected dy^{3K}/dy^{3K} after 14 days (n=6, 4 and 5, respectively). Data are expressed in arbitrary units (AU) as laminin □4 is normalized to α-actinin. *, p<0.05.

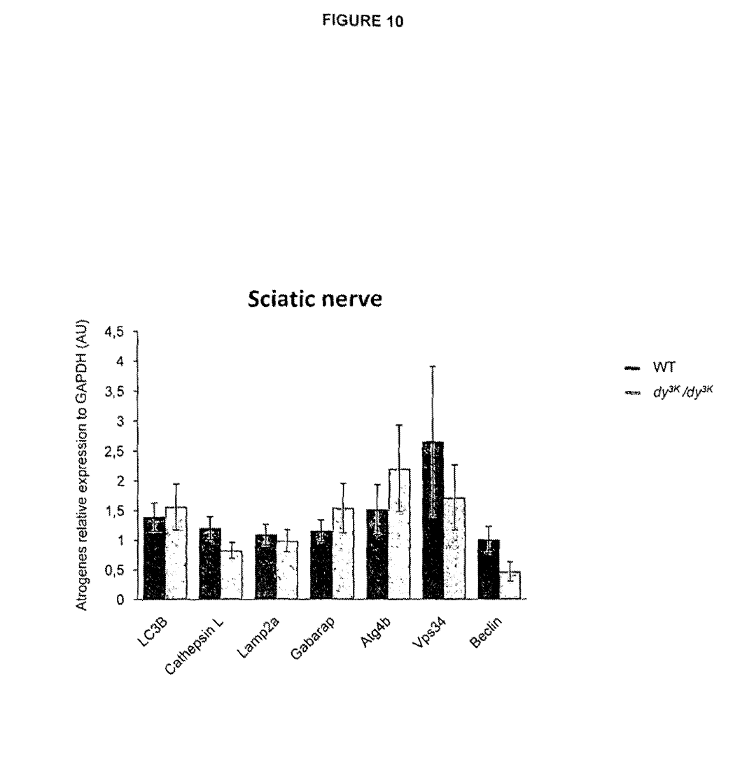

FIG. 10. Atrogene expression is not significantly modified in laminin α2 deficient peripheral nerve. Relative amounts of LC3B, Cathepsin L, Lamp2a, Gabarapl1, Atg4b, Vps34 and Beclin mRNAs in 3.5-week-old wild-type (n=5) and dy^{3K}/dy^{3K} mice (n=5). The GAPDH gene expression served as a reference.

### EXAMPLES

Congenital muscular dystrophy with laminin α2 chain deficiency (also known as MDC1A) is a severe and incapacitating disease. It has recently been shown that increased proteasomal activity is a feature of this disorder. The autophagy-lysosome pathway is the other major system involved in degradation of proteins and organelles within the muscle cell. However, it remains to be determined if the autophagy-lysosome pathway is overactive in muscular dystrophies including MDC1A. Using the dy3K/dy3K mouse model of laminin α2 chain deficiency and MDC1A patient muscle cells, it is now shown that expression of autophagy-related genes is upregulated in laminin α2 chain deficient muscle. Moreover, it is found that autophagy inhibition significantly improves the dystrophic dy3K/dy3K phenotype. In particular, it is shown that systemic injection of 3-methyladenine (3-MA) reduces muscle fibrosis, atrophy, apoptosis and increases muscle regeneration and weight. Importantly, lifespan and locomotive behaviour were also greatly improved. These findings demonstrate that enhanced autophagic activity is pathogenic and that autophagy inhibition has therapeutic potential in the treatment of MDC1A.

### Introduction

Macroautophagy (hereafter referred to as autophagy or autophagocytosis) is a multi-step catabolic process involving the sequestration of bulk cytoplasm, long-lived proteins and cellular organelles in autophagosomes, which are subsequently fused with lysosomes and content is digested by lysosomal hydrolases (1, 2). Autophagy is generally activated by conditions of nutrient or growth factor deprivation as well as endoplasmic reticulum stress. In addition, autophagy has also been associated with a number of physiological processes including development, differentiation, or pathologies like neurodegenerative diseases, lysosomal storage diseases, infection, or cancer (1,3). However, very little is known about autophagy and muscular dystrophy. The role and regulation of the autophagic pathway in skeletal muscle is still largely unknown but it is believed that excessive autophagy activation contributes to muscle loss in different catabolic conditions (4). Interestingly, inhibition of the autophagic flow may also result in muscle atrophy (5). In yeast, autophagy is controlled by more than 30 autophagy-related genes and many of them have mammalian homologues (6). Notably, through inhibition of Akt, FoxO3 controls the transcription of autophagy-related genes (e.g. LC3, Cathepsin L, Lamp2a, Gabarapl1, Vps34, Atg4b and Beclin) and therefore the autophagic-lysosomal pathway during muscle atrophy (7-9).

Recently, it was demonstrated that autophagy is impaired in collagen VI deficient muscular dystrophy and that its reactivation ameliorated the dystrophic phenotype in a mouse model of the disease (10). Another type of congenital muscular dystrophy is MDC1A (OMIM #607855), which is caused by autosomal recessive mutations in the human LAMA2 gene, encoding the α2 subunit of the extracellular basement membrane protein laminin-211. MDC1A is characterized by severe generalized muscle weakness, joint contractures and peripheral neuropathy. Around 30% of the patients die within their first decade of life (11,12). The generated null mutant dy3K/dy3K mouse model for laminin α2 chain deficiency recapitulates human disease and presents severe muscular dystrophy and dy3K/dy3K mice also display peripheral neuropathy (13, 14). Histological features of laminin α2 chain deficient muscles include degeneration/regeneration cycles, fiber size variability, apoptosis and marked connective tissue proliferation. Also, skeletal muscle atrophy is a prevalent feature of MDC1A (11, 12, 15).

In the following study it is shown that expression of autophagy-related genes is upregulated in laminin α2 chain deficient muscle and that inhibition of the autophagy process significantly improves the dystrophic phenotype in the dy3K/dy3K mouse model.

### Materials & Methods

### Transgenic Animals

Laminin α2 chain deficient mice (dy3K/dy3K), which lack laminin α2 chain completely, were used and previously described (13, 20). These mice develop severe muscular dystrophy and peripheral neuropathy and the median survival is around 22 days. For all experiments, dy3K/dy3K mice were compared with their wild-type (WT) littermates. Animals were maintained in the animal facilities of Biomedical Center (Lund) according to animal care guidelines, and permission was given by the regional ethical board.

### Primary Muscle Cell Culture and Differentiation

Primary myoblasts were obtained from a control fetus (12 weeks of gestation) and a MDC1A fetus (15 weeks of gestation), presenting a homozygous nonsense mutation in exon 31 of the LAMA2 gene. Muscle cells were obtained in accordance with the French legislation on ethical rules.

Cells were cultivated in 6-well plates with growth medium (F10-Ham medium, Gibco) containing 20% foetal bovine serum (Gibco) at 37° C., 5% CO₂. At about 70% confluency, differentiation into myotubes was initiated by switching to fusion medium (DMEM, Gibco) containing 2% horse serum (Gibco), 10⁻⁶ M insulin (Sigma) and 2.5×10⁻⁶ M dexamethasone (Sigma). Protein lysates were obtained by scrapping the cells directly into the lysis buffer (50 mM Tris-HCl, pH 6.8, 10% 13-mercaptoethanol, 4% SDS, 0.03% bromophenol blue and 20% glycerol).

### Systemic Injections of 3-Methyladenine

Systemic administration was performed by intraperitoneal injection of 3-MA (15 mg/kg) into dy3K/dy3K mice and control littermates at the age of 2.5 weeks and 3.5 weeks. Mice were sacrificed 14 days after injection and quadriceps and tibialis anterior muscles were processed for morphometric analysis, immunofluorescence experiments, qRT-PCR or Western blot analysis. Prior to the euthanasia, an exploratory locomotion test was performed.

### RNA Extraction, Reverse Transcription and Quantitative Real-Time PCR

Total RNA was extracted from 10 mg quadriceps muscle of 6 dy3K/dy3K mice (3.5-week-old) and 6 WT littermates and 5 WT and 5 dy3K/dy3K mice treated with 3-MA using RNeasy mini kit (Qiagen) including an initial step of proteinase K digestion (Fermentas, 240 ng/μl). Complementary DNA was synthesized from 1 μg of total RNA with random primers and SuperScriptlll reverse transcriptase (Invitrogen) following manufacturer's instructions. Quantitative PCRs were performed in triplicate with the Maxima SYBR Green qPCR Master Mix (Fermentas). Expression of target and reference genes was monitored using a real-time qRT-PCR method (Light Cycler, Roche) with the previously described primers for the autophagic genes LC3B, Cathepsin L, Lamp2a, Gabarapl1, Atg4b, Vps34 and Beclin (8). The amplification efficiency for each primer pair was evaluated by amplification of serially diluted template cDNAs (E=10^{-r/slope}). Efficiency corrected RNA levels (in arbitrary units) were calculated by using the formula E^{-Ct}. Expression levels were then calculated relative to the endogenous control gene GAPDH and relative to wild-type quadriceps.

### Protein Extraction and Western Blot Analyses

Isolated quadriceps muscles were obtained from 6 wild-type, 6 dy3K/dy3K mice (3.5 weeks of age) and 6 dy3K/dy3K mice 48 h or 14 days after 3-MA injection. Each sample was immediately frozen in liquid nitrogen and reduced to powder using a mortar. Protein extracts were obtained as previously described (16). A total of 30 pg of denaturated protein was loaded on 10-20% acrylamide SDS-gels (Clearpage, CBS Scientific) and blotted onto nitrocellulose membranes (Hybond-C, Amersham) during 1.5 hour (Biorad). The membranes were blocked for one hour at RT in PBS, 0.01% Tween-20, 5% milk and incubated overnight at 4° C. with rabbit polyclonal antibodies directed against pAkt (Ser 473, 1/2000, #4060 or Thr 308, 1/1000, #2965, Cell Signaling Technology), Akt (1/1000, #4685, Cell Signaling Technology), Vps34 (1/200, V9764, Sigma) or LC3B (1/250, #2775, Cell Signaling Technology). Blots were then washed 3 times 10 minutes with PBS, 0.05% Tween 20, incubated with horseradish peroxidase-conjugated polyclonal goat anti-rabbit (1/4000, sc-2004, Santa Cruz Biotechnology) or goat anti-mouse (1/4000, sc-2005, Santa Cruz Biotechnology) antibody for 1 hour. Membranes were incubated in ECL (Amersham Biosciences), exposed on Hyperfilm (Amersham Biosciences) and developed (AGFA, Curix 60). Each membrane was rehybridized with mouse monoclonal anti-tubulin (1/4000, clone DM 1A, Sigma) for loading normalization.

The quantifications were performed using ImageJ 1.40 (http://rsb.info.nih.gov/ij/download.html).

### Histology and Immunofluorescence Experiments

Quadriceps and tibialis anterior muscles from wild-type, dy3K/dy3K and injected mice (n=6 for each group) were rapidly dissected after euthanasia and frozen in OCT (Tissue Tek) in liquid nitrogen. Serial sections of 7 pm were either stained with hematoxylin and eosin or processed for immunofluorescence experiments following standard procedures (20) with rabbit polyclonal antibodies directed against LC3B (1/100, #3868, Cell Signaling Technology), laminin γ1 chain (1/1000, #1083), laminin α4 chain (1/400, #1100) and laminin 32 chain (1/400, #1117) generously provided by Dr. T. Sasaki, rat monoclonal antibodies against laminin γ1 chain (1/200, MAB 1914, Chemicon) and tenascin-C (undiluted, MTn15), goat polyclonal antibody against collagen III (1/100, #1330, SouthernBiotech) and mouse monoclonal antibodies against caspase-3 (1/100, CPP32, BD Transduction Laboratory), embryonic myosin heavy chain (F1.652, Developmental Studies Hybridoma Bank) and MyoD (1/100, clone 5.8A Dako). For apoptotic myofiber detection, a TUNEL detection kit was used following instructions of the manufacturer (GenScript). Sections were analyzed using a Zeiss Axioplan fluorescence microscope. Images were captured using an ORCA 1394 ER digital camera with the Openlab 3 software.

### Exploratory Locomotion Test

Exploratory locomotion was examined in an open field test. In each experiment, the mouse 14 days after 3-MA injection (n=11 for dy3K/dy3K and wild-type, respectively) was placed into a new cage and allowed to explore the cage for 5 min. The time that the mouse spent moving around was measured manually.

### Survival Curves

Death was monitored in 3-MA injected dy3K/dy3K mice (n=9). A survival curve was constructed using the GraphPad Prism 4 software.

### Morphometric Analysis

Measurements were performed on whole quadriceps or tibialis anterior muscle sections from untreated wild-type and dy3K/dy3K, wild-type and dy3K/dy3K 3-MA injected animals (n=6 for each group). Tenascin-C and collagen III positive areas, eMHC positive fibers, caspase-3 positive fibers, TUNEL-positive myonuclei and fiber diameters were measured using the imageJ software. Minimal Feret's diameter was measured (41) for at least 1500 fibers for each mouse. The same number of fibers was used for quantification of fibers with centrally located nuclei. Wet quadriceps muscle weights were determined from 7 uninjected wild-type or dy3K/dy3K and 3-MA treated wild-type (n=4) and dy3K/dy3K (n=6) animals and correlated to body weight.

### Statistical Analysis

All tests for analysis of significance were done using the GraphPad Prism 4 software. For quantitative PCR experiments, protein quantifications, morphometric analysis and exploratory locomotion test, one way ANOVA followed by a Bonferroni's post multiple comparison test was performed. Regarding fiber size distribution, a X2-test was calculated and paired comparison of distribution was estimated related for a p-value inferior to 0.0001. Finally, statistic LogRank test was used for analysis of significance of survival curves. Data always represent mean±SEM.

### Results

### Increased Expression of Autophagy Related Genes in Laminin α2 Chain-Deficient Muscle

To determine whether the activity of the autophagy lysosome pathway is increased in laminin α2 chain deficient muscle, we first analyzed the expression of members of this pathway in dy3K/dy3K animals and in particular those controlled by the transcription FoxO3, whose expression is increased approximately 2-fold in dy3K/dy3K animals (16). We detected significantly increased mRNA levels of the microtubule-associated protein-1 light chain 3B (LC3B) in quadriceps muscles from dy3K/dy3K mice (FIG. 1A). LC3B is one of the three (human) LC3 isoforms that undergoes post-translational modifications during autophagy. The presence of LC3 in autophagosomes and the conversion of LC3 to the lower migrating form LC311 have been used as indicators of autophagy (17, 18). By immunofluorescence analysis we detected accumulated LC3B in dy3K/dy3K quadriceps muscle fibers (FIG. 1B) and Western blot analysis revealed an approximate 2-fold increase of LC3B11 expression in dy3K/dy3K quadriceps muscle (FIG. 1C). Similarly, we noted an increased mRNA expression of the autophagosome membrane markers Gabarapl1, Beclin and Vps34 as well as the cysteine protease Atg4B in dy3K/dy3K quadriceps muscle (FIG. 1A). Finally, mRNA expression of lysosomal markers Cathepsin L and Lamp2a was also significantly increased in dy3K/dy3K quadriceps muscle.

To determine if enhanced expression of autophagy-related genes also is seen in human laminin α2 chain deficient muscle, we analyzed primary myoblasts and myotubes from a control and a laminin α2 chain deficient patient. Increased protein expression of LC3BII, Vps34, Cathepsin L and Beclin was noted in the MDC1A myotubes but not in corresponding myoblasts (FIG. 1D).

Laminin α2 chain interacts with the dystrophin-glycoprotein and mutations in several of its components lead to various forms of muscular dystrophy (19). To investigate if autophagy is modified when dystrophin is absent and other members of the dystrophin-glycoprotein complex are reduced, we quantified the expression level of autophagy related genes in quadriceps from mdx mice (a Duchenne muscular dystrophy mouse model). We found no major modification in the expression of LC3B, Gabarapl1, Beclin, Vps34 and Atg4B mRNAs in 5-week- or 3-month-old mice. Only Cathepsin L mRNA expression was elevated in 5-week- and 3-month-old mdx muscle and Lamp-2 mRNA expression was also increased in 3-month-old mdx mice (FIG. 8), suggesting that microautophagy followed by chaperone-mediated autophagy could be modified in this disease.

### Systemic Injection of 3-Methyladenine (3-MA) Restores Autophagic Gene Expression in Laminin α2 Chain-Deficient Muscle

Since the autophagy-lysosome pathway system seemed to be overactive in dy3K/dy3K muscle, we envisaged that inhibition of the autophagy pathway could improve muscle shape and mouse physiology. Thus, we administered the autophagy inhibitor 3-MA into the peritoneum of 2.5-week-old dy3K/dy3K mice. At this age, the dy3K/dy3K mice start to be distinguishable from their littermates. We repeated the injection at 3.5 weeks of age.

The median survival of dy3K/dy3K mice is around 22 days and most if not all dy3K/dy3K are dead by 4 weeks of age (16). We analyzed mice and muscles 14 days post-injection (a time point when dy3KIdy3K mice should be dead). Notably, we found that the systemic injection of 3-MA restored the expression of the autophagy-related genes to the basal level (FIGS. 1A-C). Systemic injection of 3-MA improves muscle morphology in laminin α2 chain-deficient muscle Remarkably, the 3-MA injections resulted in considerably improved muscle morphology.

We first evaluated the main histological hallmarks of the dystrophic process (pathological fibrosis and muscle fiber diameter) by morphometric measurements. Collagen III expression, which previously has been shown to be increased in dy3 Kdy3K muscle (16), was reduced in 3-MA injected mice compared with non-injected dy3K/dy3K mice (FIG. 2B). To further confirm the reduction of fibrosis in 3-MA-treated animals, we analyzed tenascin-C expression, which also has been demonstrated to be increased in dy3K/dy3K muscle (16, 20). Similarly, tenascin-C expression was reduced in 3-MA injected mice compared with non-injected dy3K/dy3K mice (FIG. 2B).

We also investigated the expression of laminin α4 and 132 chains in 3-MA treated dy3K/dy3K mice. It has previously been shown that the expression of laminin α4 chain is increased at the dy3K/dy3K sarcolemma whereas the laminin 32 chain expression is reduced (20, 21). Expression of both proteins was near normal in injected mice (FIG. 9).

It is well established that the average fibre diameter is significantly reduced in dy3K/dy3K muscle (16, 22, 23). Notably, the average fibre diameter was increased upon 3-MA injection and fibre size distribution in quadriceps muscle was significantly shifted towards larger fibres for both wild-type and dy3K/dy3K injected animals (FIGS. 3A, B). We observed that 25% of dy3K/dy3K quadriceps fibres have a diameter inferior to 26 pₘ, whereas the number is about 15% in wild-type and dy3K/dy3K injected animals, respectively. Furthermore, the ratio of quadriceps muscle wet weight per body weight was normalized in 3-MA injected dy3K/dy3K mice, compared to age-matched non-injected dy3K/dy3K mice (FIG. 3C).

### Systemic Injection of 3-MA Stimulates Muscle Regeneration in Laminin α2 Chain-Deficient Muscle

The proportion of centrally-located nuclei is one of the main features of the degeneration-regeneration process. The number of cells with centrally located nuclei was slightly but significantly elevated in 3-MA injected dy3K/dy3K mice (FIG. 4A). We additionally performed immunofluorescence experiments analyzing the expression of regeneration markers embryonic myosin heavy chain (a specific marker of newly regenerated fibers) and MyoD1 (present in activated satellite cells and myoblasts). Indeed, the proportion of fibers expressing eMHC significantly increased with the 3-MA injection of dy3K/dy3K mice (FIG. 4B). Also, the amount of MyoD1 positive nuclei was increased in 3-MA injected dy3K/dy3K mice (FIG. 4C).

Apoptosis is Decreased after Systemic Injection of 3-MA

As apoptosis contributes to the disease progression, we analyzed the apoptosis rate occurring in skeletal muscle of systemically injected mice. As previously described, the number of caspase-3 positive fibers (containing caspase-3 and pro-caspase 3 proteins) in dy3K/dy3K mice, was significantly increased when compared to controls (16). Forty-eight hours after 3-MA injection, we were able to find caspase-3 positive fibers in the same proportion as in non-injected dy3K/dy3K mice (data not shown). However, 14 days after injection the proportion of caspase 3 positive fibers was significantly decreased in 3-MA injected dy3K/dy3K quadriceps (FIG. 5A-B). These results were further confirmed using the TUNEL enzymatic labeling assay. We found that the proportion of TUNEL-positive myonuclei was significantly reduced in 3-MA treated dy3KIdy3K animals (FIG. 5C).

### Systemic Injection of 3-MA Restores Akt Phosphorylation

We have recently demonstrated that Akt phosphorylation on both threonine 308 and serine 473 is diminished in dy3K/dy3K quadriceps muscle, whereas the total level of Akt is unchanged (16). To investigate whether injection of 3-MA could restore Akt activity, we sacrificed mice 48 h and 14 days after injection and learned that Akt phosphorylation on both sites was restored to wild-type levels at both time points (FIG. 6A-B).

Systemic Injection of 3-MA Increases Survival and Locomotive Behaviour, but does not Significantly Improve Peripheral Neuropathy

Dy3K/dy3K mice are significantly less active in an open field test (16). Remarkably, 3-MA injected dy3K/dy3K mice displayed the same level of activity as wild-type animals (FIG. 357A). Also, 3-MA treated dy3K/dy3K mice weighed significantly more than non-injected dy3K/dy3K mice, although they never reached the weight of wild-type mice (FIG. 7B).

Moreover, the median survival of 3-MA injected dy3K/dy3K mice was 37 days (FIG. 7C), whereas it has been shown to be 22 days for non-treated dy3K/dy3K mice (16). Finally, although survival and muscle morphology was significantly improved, transient hind leg paralysis often occurred in one leg of 3-MA treated dy3K/dy3K mice and similar paralysis occurred in non-treated dy3K/dy3K mice but not in 3-MA injected wild-type mice (16) (data not shown). Yet, this transient paralysis of had no obvious effect on the locomotive behavior. Nevertheless, it is clear that 3-MA did not appreciably improve the pathology of the peripheral nerve. In agreement with this observation, we found no increased mRNA levels of autophagy-related genes in laminin α2 chain deficient sciatic nerve (FIG. 10).

### Discussion

MDC1A is a debilitating muscle disease for which there currently is no cure. Several approaches to prevent disease in MDC1A mouse models have been explored and they include gene replacement—(20, 24, 25), anti-apoptosis—(26-28), proteasome inhibition-(16), cell—(29) and improved regeneration therapy (30). While the transgenic strategies (e.g. over-expression of laminin α2 chain, mini-agrin and in particular laminin α1 chain) may have offered the most complete muscle restoration they are not yet clinically feasible and the pharmacological inhibition of apoptosis and proteasome, respectively, have only resulted in partial recovery. Hence, other potential therapeutic targets should be explored. Here we present data indicating that increased autophagy is pathogenic in MDC1A. We found increased expression of several autophagy-related genes in laminin α2 chain deficient mouse and human muscle cells. We have shown that autophagy inhibition, using 3-MA in the dy3K/dy3K mouse model for MDCIA, significantly reduces many of the pathological symptoms in the dystrophic mice.

Apoptosis has been described as a major feature in MDC1A and its inhibition by genetic or pharmacological therapy ameliorated several pathological symptoms in the dyW/dyW mouse model of MDC1A (26-28, 31). Autophagy and apoptosis are interconnected by common proteins and functions. First, autophagy is a basal mechanism for elimination of damaged protein or organelles. Therefore accumulation of mitochondria or misfolded proteins could initiate oxidative stress and cell death. Second, it has recently been described that the regulation of autophagy by survival signals in skeletal muscle is controlled by a rapid transcription-independent mechanism through mTOR, and a long term but more effective transcription program requiring FoxO3 (7-9, 32). Finally, anti-apoptotic proteins, such as Bcl-2 family members, inhibit Beclin-1 and induction of autophagy proteins could enhance cell death (33, 34). Consequently, it would be interesting to test whether the combined inhibition of apoptosis and autophagy would further restore the phenotype of laminin α2 chain deficient mice. Furthermore, we recently showed that global ubiquitination of proteins is raised in dy3K/dy3K muscles and that proteasome inhibition improves the dystrophic phenotype (16). In addition, it has been demonstrated that ubiquitinated proteins also can be delivered to the autophagosomes through the p62/SQSTM1 complex that is able to bind LC3 (35-39). Hence, we would like to evaluate combinatorial treatment of autophagy and proteasome inhibition.

Interestingly, together with the data we presented here, incorrect function of autophagy has been discovered to be pathogenic in the two most common forms of congenital muscular dystrophy and both are linked to deficiency of extracellular matrix proteins (10). We therefore hypothesize that an extracellular matrix unbalance affects the autophagy pathway. The additional data that we provide on the Duchenne mouse model mdx, showing that autophagy is not modified, reinforces this hypothesis. In this model, it seems that microautophagy followed by chaperone-mediated autophagy (dependant of Lamp2) could be stimulated with the progression of the disease. This should be further clarified as well as the potential primary or secondary contribution of autophagy in other congenital muscle diseases (dystroglycanopathies or congenital myopathies). Autophagosomes are present in many myopathies and are the major features of a group of muscle disorders named autophagic vacuolar myopathies. This group is composed by the late-onset Pompe disease, caused by a defect in lysosomal acid maltase (MIM ID #232300), Danon disease that primarily affects the heart, due to a defect in the LAMP2 gene (MIM ID #300257), and X-linked myopathy with excessive autophagy (XMEA), associated with mutations in the VMA21 gene (40). Therefore, autophagy related genes could be potential candidate genes mutated in genetically irresolute muscle diseases.

In summary, our study demonstrates for the first time that autophagy can be overactive in a congenital muscular dystrophy condition. In addition, its inhibition improves the muscle phenotype of laminin α2 chain deficient mice.

The results provide compelling evidence in support of the efficacy of autophagy inhibitors in the treatment and prevention of muscular dystrophies, such as MDC1A.

### REFERENCES

1. Eskelinen, E. L., and Saftig, P. 2009. Autophagy: a lysosomal degradation pathway with a central role in health and disease. Biochim Biophys Acta 1793:664-673.
2. Kundu, M., and Thompson, C. B. 2008. Autophagy: basic principles and relevance to disease. Annu Rev Pathol 3:427-455.
3. Mizushima, N., Levine, B., Cuervo, A. M., and Klionsky, D. J. 2008. Autophagy fights disease through cellular self-digestion. Nature 451:1069-1075.
4. Sandri, M. Autophagy in skeletal muscle. FEBS Lett 584:1411-1416.
5. Masiero, E., Agatea, L., Mammucari, C., Blaauw, B., Loro, E., Komatsu, M., Metzger, D., Reggiani, C., Schiaffino, S., and Sandri, M. 2009. Autophagy is required to maintain muscle mass. Cell Metab 10:507-515.
6, Lum, J. J., DeBerardinis, R. J., and Thompson, C. B. 2005. Autophagy in metazoans: cell survival in the land of plenty. Nat Rev Mol Cell Biol 6:439-448.
7. Mammucari, C., Milan, G., Romanello, V., Masiero, E., Rudolf, R., Del Piccolo, P., Burden, S. J., Di Lisi, R., Sandri, C., Zhao, J., et al. 2007. FoxO3 controls autophagy in skeletal muscle in vivo. Cell Metab 6:458-471.
8. Zhao, J., Brault, J. J., Schild, A., Cao, P., Sandri, M., Schiaffino, S., Lecker, S. H., and Goldberg, A. L. 2007. FoxO3 coordinately activates protein degradation by the autophagic/lysosomal and proteasomal pathways in atrophying muscle cells. Cell Metab 6:472-483.
9. Zhao, J., Brault, J. J., Schild, A., and Goldberg, A. L. 2008. Coordinate activation of autophagy and the proteasome pathway by FoxO transcription factor. Autophagy 4:378-380.
10. Grumati, P., Coletto, L, Sabatelli, P., Cescon, M., Angelin, A., Bertaggia, E., Blaauw, B., Urciuolo, A., Tiepolo, T., Merlini, L., et al. Autophagy is defective in collagen VI muscular dystrophies, and its reactivation rescues myofiber degeneration. Nat Med 16:1313-1320.
11. Voit T, and Tome F M. 2004. The congenital muscular dystrophies. Myology Third edition, Engel A G and Franzini-Armstrong C eds, McGrawHill II: 1203-1238.,
12. Tome, F. M., Evangelista, T., Leclerc, A., Sunada, Y., Manole, E., Estournet, B., Barois, A., Campbell, K. P., and Fardeau, M. 1994. Congenital muscular dystrophy with merosin deficiency. C R Acad Sci III 317:351-357.
13. Miyagoe, Y., Hanaoka, K., Nonaka, I., Hayasaka, M., Nabeshima, Y., Arahata, K., and Takeda, S. 1997. Laminin alpha2 chain-null mutant mice by targeted disruption of the Lama2 gene: a new model of merosin (laminin 2)-deficient congenital muscular dystrophy. FEBS Lett 415:33-39.
14. Nakagawa, M., Miyagoe-Suzuki, Y., Ikezoe, K., Miyata, Y., Nonaka, I., Harii, K., and Takeda, S. 2001. Schwann cell myelination occurred without basal lamina formation in laminin alpha2 chain-null mutant (dy3K/dy3K) mice. Glia 35:101-110.
15. Allamand, V., and Guicheney, P. 2002. Merosin-deficient congenital muscular dystrophy, autosomal recessive (MDC1A, MIM#156225, LAMA2 gene coding for alpha2 chain of laminin). Eur J Hum Genet. 10:91-94.
16. Carmignac, V., Quere, R., and Durbeej, M. Proteasome inhibition improves the muscle of laminin alpha2 chain-deficient mice. Hum Mol Genet. 20:541-552.
17. Levine, B., and Kroemer, G. 2008. Autophagy in the pathogenesis of disease. Cell 132:27-42.
18. Maiuri, M. C., Zalckvar, E., Kimchi, A., and Kroemer, G. 2007. Self-eating and self-killing: crosstalk between autophagy and apoptosis. Nat Rev Mol Cell Biol 8:741-752.
19. Barresi, R., and Campbell, K. P. 2006. Dystroglycan: from biosynthesis to pathogenesis of human disease. J Cell Sci 119:199-207.
20. Gawlik, K., Miyagoe-Suzuki, Y., Ekblom, P., Takeda, S., and Durbeej, M. 2004. Laminin alpha1 chain reduces muscular dystrophy in laminin alpha2 chain deficient mice. Hum Mol Genet. 13:1775-1784.
21. Cohn, R. D., Herrmann, R., Wewer, U. M., and Voit, T. 1997. Changes of laminin beta 2 chain expression in congenital muscular dystrophy. Neuromuscul Disord 7:373-378.
22. Gawlik K I, A. M., Carmignac V, Elamaa H, Durbeej M. 2010. Distinct Roles for Laminin Globular Domains in Laminin al Chain Mediated Rescue of Murine Laminin α2 Chain Deficiency. In PLoS ONE e11549.
23. Bentzinger, C. F., Barzaghi, P., Lin, S., and Ruegg, M. A. 2005. Overexpression of mini-agrin in skeletal muscle increases muscle integrity and regenerative capacity in laminin-alpha2-deficient mice. FASEB J 19:934-942.
24. Kuang, W., Xu, H., Vachon, P. H., Liu, L., Loechel, F., Wewer, U. M., and Engvall, E. 1998. Merosin-deficient congenital muscular dystrophy. Partial genetic correction in two mouse models. J Clin Invest 102:844-852.
25. Meinen, S., Barzaghi, P., Lin, S., Lochmuller, H., and Ruegg, M. A. 2007. Linker molecules between laminins and dystroglycan ameliorate laminin-alpha2-deficient muscular dystrophy at all disease stages. J Cell Biol 176:979-993.
26. Erb, M., Meinen, S., Barzaghi, P., Sumanovski, L. T., Courdier-Fruh, I., Ruegg, M. A., and Meier, T. 2009. Omigapil ameliorates the pathology of muscle dystrophy caused by laminin-alpha2 deficiency. J Pharmacol Exp Ther 331:787-795.
27. Girgenrath, M., Dominov, J. A., Kostek, C. A., and Miller, J. B. 2004. Inhibition of apoptosis improves outcome in a model of congenital muscular dystrophy. J Clin Invest 114:1635-1639.
28. Girgenrath, M., Beermann, M. L., Vishnudas, V. K., Homma, S., and Miller, J. B. 2009. Pathology is alleviated by doxycycline in a laminin-alpha2-null model of congenital muscular dystrophy. Ann Neurol 65:47-56.
29. Hagiwara, H., Ohsawa, Y., Asakura, S., Murakami, T., Teshima, T., and Sunada, Y. 2006. Bone marrow transplantation improves outcome in a mouse model of congenital muscular dystrophy. FEBS Lett 580:4463-4468.
30. Kumar, A., Yamauchi, J., Girgenrath, T., and Girgenrath, M. 2011. Muscle-specific expression of insulin-like growth factor 1 improves outcome in Lama2Dy-w mice, a model for congenital muscular dystrophy type 1A. Hum Mol Genet Epub ahead of print
31. Dominov, J. A., Kravetz, A. J., Ardelt, M., Kostek, C. A., Beermann, M. L., and Miller, J. B. 2005. Muscle-specific BCL2 expression ameliorates muscle disease in laminin {alpha}2-deficient, but not in dystrophin-deficient, mice. Hum Mol Genet. 14:1029-1040.
32. Mammucari, C., Schiaffino, S., and Sandri, M. 2008. Downstream of Akt: FoxO3 and mTOR in the regulation of autophagy in skeletal muscle. Autophagy 4:524-526.
33. Pattingre, S., Tassa, A., Qu, X., Garuti, R., Liang, X. H., Mizushima, N., Packer, M., Schneider, M. D., and Levine, B. 2005. Bcl-2 antiapoptotic proteins inhibit Beclin 1-dependent autophagy. Cell 122:927-939.
34. Scott, R. C., Juhasz, G., and Neufeld, T. P. 2007. Direct induction of autophagy by Atg1 inhibits cell growth and induces apoptotic cell death. Curr Biol 17:1-11.
35. Ichimura, Y., Kominami, E., Tanaka, K., and Komatsu, M. 2008. Selective turnover of p62/A170/SQSTM1 by autophagy. Autophagy 4:1063-1066.
36. Ichimura, Y., Kumanomidou, T., Sou, Y. S., Mizushima, T., Ezaki, J., Ueno, T., Kominami, E., Yamane, T., Tanaka, K., and Komatsu, M. 2008. Structural basis for sorting mechanism of p62 in selective autophagy. J Biol Chem 283:22847-22857.
37. Kirkin, V., McEwan, D. G., Novak, I., and Dikic, i. 2009. A role for ubiquitin in selective autophagy. Mol Cell 34:259-269.
38. Komatsu, M., Waguri, S., Koike, M., Sou, Y. S., Ueno, T., Hara, T., Mizushima, N., Iwata, J., Ezaki, J., Murata, S., et al. 2007. Homeostatic levels of p62 control cytoplasmic inclusion body formation in autophagy-deficient mice. Cell 131:1149-1163.
39. Pankiv, S., Clausen, T. H., Lamark, T., Brech, A., Bruun, J. A., Outzen, H., Overvatn, A., Bjorkoy, G., and Johansen, T. 2007. p62/SQSTM1 binds directly to Atg8/LC3 to facilitate degradation of ubiquitinated protein aggregates by autophagy. J Biol Chem 282:24131-24145.
40. Ramachandran, N., Munteanu, I., Wang, P., Aubourg, P., Rilstone, J. J., Israelian, N., Naranian, T., Paroutis, P., Guo, R., Ren, Z. P., et al. 2009. VMA21 deficiency causes an autophagic myopathy by compromising V-ATPase activity and lysosomal acidification. Cell 137:235-246.
41. Briguet, A., Courdier-Fruh, I., Foster, M., Meier, T., and Magyar, J. P. 2004. Histological parameters for the quantitative assessment of muscular dystrophy in the mdx-mouse. Neuromuscul Disord 14:675-682.

1-54. (canceled)

55. A method for treating or preventing muscular dystrophy in a mammal comprising administering an effective amount of an autophagy inhibitor to the mammal.

56. The method of claim 55, wherein the autophagy inhibitor is a macroautophagy inhibitor.

57. The method of claim 55, wherein the autophagy inhibitor is selected from the group consisting of 3-methyladenine, wortmannin, bafilomycins (such as bafilomycin A1), chloroquine, hydroxychloroquine, PI3K class III inhibitors (such as LY294002), L-asparagine, catalase, E64D, leupeptin, N-acetyl-L-cysteine, pepstatin A, propylamine, 4-aminoquionolines, 3-methyl adenosine, adenosine, okadaic acid, N6-mercaptopurine riboside (N-6-MPR), an aminothiolated adenosine analogue and 5-amino-4-imidazole carboxamide riboside (AICAR).

58. The method of claim 55, wherein the muscular dystrophy is selected from the group consisting of congenital muscular dystrophy, Duchenne muscular dystrophy (DMD), Becker's muscular dystrophy (BMD, Benign pseudohypertrophic muscular dystrophy), distal muscular dystrophy (distal myopathy), Emery-Dreifuss muscular dystrophy (EDMD), facioscapulohumeral muscular dystrophy (FSHMD, FSHD or FSH), limb-girdle muscular dystrophy (LGMD), myotonic muscular dystrophy, centronuclear myopathies and oculopharyngeal muscular dystrophy.

59. The method of claim 58, wherein the muscular dystrophy is a congenital muscular dystrophy selected from the group consisting of:
(a) Congenital muscular dystrophy with abnormalities in the extracellular matrix, such as Merosin (laminin α2) deficient CMD (MDCIA) and Collagen VI deficient CMD (Ullrich CMD and Bethlem myopathy);
(b) Dystroglycanopathies (abnormalities of α-dystroglycan), such as Fukuyama-type CMD, Variants of muscle-eye brain disease, Walker-Warburg syndrome, Congenital muscular dystrophy type 1C, Congenital muscular dystrophy type 1D and Limb-girdle muscular dystrophy 21;
(c) Defects in the integrin α7 subunit, such as Congenital myopathy with integrin α7 deficiency;
(d) Abnormalities of nuclear envelope proteins, such as L-CMD;
(e) Abnormalities in ER, such as SEPN1 related myopathy (formerly known as Rigid Spine Muscular Dystrophy);
(f) Undiagnosed CMD, including merosin positive; and
(g) Ryanodine receptor gene (RYR1) CMD

60. The method of claim 59, wherein the muscular dystrophy is laminin-α2-deficient congenital muscular dystrophy (Muscular Dystrophy, Congenital Merosin-Deficient, 1a/MDCIA).

61. The method of claim 59, wherein the muscular dystrophy is not laminin-α2-deficient congenital muscular dystrophy (Muscular Dystrophy, Congenital Merosin-Deficient, 1a/MDCIA).

62. The method of claim 58, wherein the muscular dystrophy is Duchenne muscular dystrophy (DMD).

63. The method of claim 58, wherein the muscular dystrophy is a distal muscular dystrophy (distal myopathy)-selected from the group consisting of Miyoshi myopathy, distal myopathy with anterior tibial onset, and Welander distal myopathy.

64. The method of claim 58, wherein the muscular dystrophy is an Emery-Dreifuss muscular dystrophy (EDMD) selected from the group consisting of EDMD1, EDMD2, EDMD3, EDMD4, EDMD5 and EDMD6.

65. The method of claim 58, wherein the muscular dystrophy is a facioscapulohumeral muscular dystrophy (FSHMD, FSHD or FSH) from the group consisting of FSHMD1A (4q35 deletion) and FSHMDIB.

66. The method of claim 58, wherein the muscular dystrophy is a Limb-girdle muscular dystrophy or (Erb's muscular dystrophy selected from the group consisting of LGMD1A, LGMD1B, LGMD1C, LGMD1D, LGMD1E, LGMD1F, LGMD1G, LGMD2A, LGMD2B, LGMD2C, LGMD2D, LGMD2E, LGMD2F, LGMD2G, LGMD2H, LGMD21, LGMD2J, LGMD2K, LGMD2L, LGMD2M, LGMD2N and LGMD20.

67. The method of claim 58, wherein the muscular dystrophy is a myotonic dystrophy selected from the group consisting of DM1 (also called Steinert's disease) severe congenital form, DM1 childhood-onset form and DM2 (also called proximal myotonic myopathy or PROMM).

68. The method of claim 55, wherein the muscular dystrophy is associated with excessive autophagy.

69. The method of claim 68, wherein the muscular dystrophy is associated with excessive macroautophagy.

70. The method of claim 55, wherein the inhibitor is for use in combination with a second therapeutic agent or treatment for muscular dystrophy.

71. The method of claim 70, wherein the second therapeutic agent or treatment comprises:
(a) physical therapy, corrective orthopedic surgery and/or steroids;
(b) gene replacement, cell therapy and/or anti-apoptosis therapy; and/or
(c) a proteasome inhibitor.

72. The method of claim 70, wherein the second therapeutic agent or treatment is an proteasome inhibitor.

73. The method of claim 70, wherein the autophagy inhibitor is a macroautophagy inhibitor and the proteasome inhibitor is an inhibitor of the ubiquitin-proteasome system.

74. The method of claim 55, wherein the method comprises or consists of administering an autophagy inhibitor to the mammal in combination with a proteasome inhibitor.
